SEQUENCE LISTING (5 sequences)

SEQ ID NO: 1 - protein, 214 aa, Artificial Sequence
  Description of Artificial Sequence Synthetic chimeric polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 25 aa, Cricetulus griseus
MATMVPSSVPCHWLLFLLLLFSGSS

SEQ ID NO: 3 - protein, 449 aa, Artificial Sequence
  Description of Artificial Sequence Synthetic chimeric polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 39 bp, Cricetulus griseus
atggccaccatggtgccctcttctgtgccctgccactgg

SEQ ID NO: 5 - DNA, 36 bp, Cricetulus griseus
ctgctgttcctgctgctgctgttctctggctcttct

(19) United States
(12) Patent Application Publication
(10) Pub. No.: US 2017/0247458 A1
(43) Pub. Date: Aug. 31, 2017

(54) Safety for Treating Cancers with a Glycosylated Chimeric Antibody to EGFR

(71) Applicants: Jian Cao, San Diego, CA (US); Jeffrey Su, San Diego, CA (US)

(72) Inventors: Jian Cao, San Diego, CA (US); Jeffrey Su, San Diego, CA (US)

(73) Assignee: Sorrento Therapeutics, Inc., San Diego, CA (US)

(21) Appl. No.: 15/403,102

(22) Filed: Jan. 10, 2017

Related U.S. Application Data
(60) Provisional application No. 62/276,952, filed on Jan. 10, 2016.

(51) Int. Cl. C07K 16/28

(52) U.S. Cl. CPC C07K 16/2863; C07K 2317/515; C07K 2317/24; C07K 2317/94; C07K 2317/92

(57) ABSTRACT

There is disclosed a chimeric cetuximab-like monoclonal antibody (CMAB009 mAb) having at least 80% NANA glycosylation terminal sialic acid at an N-glycosylation site Asn297 and a glycosylation pattern of Gal-α(2,3/6)-Gal. The disclosed CMAB009 monoclonal antibody is a chimeric antibody having the same amino acid sequence (light chain/heavy chain of SEQ ID NO. 1/SEQ ID NO. 3) as cetuximab (Erbitux®) which has at least 80% NGNA terminal sialic acid and a glycosylation pattern of Gal-α(1,3)-Gal.

### CROSS REFERENCE TO RELATED APPLICATION

This patent application claims priority to U.S. provisional patent application 62/276,952 filed 10 Jan. 2016.

### TECHNICAL FIELD

The present disclosure provides a chimeric cetuximab-like monoclonal antibody (CMAB009 mAb) having at least 80% NANA glycosylation terminal sialic acid at an N-glycosylation site and a glycosylation pattern of Gal-α(2,3/6)-Gal. The disclosed CMAB009 monoclonal antibody is a chimeric antibody having the same amino acid sequence (light chain/heavy chain of SEQ ID NO. 1/SEQ ID NO. 3) as cetuximab (Erbitux®). However, the disclosed antibody has at least 80% NGNA terminal sialic acid and a glycosylation pattern of Gal-α(1,3)-Gal. Therefore, the disclosed antibody demonstrates improved safety and efficacy over cetuximab.

### BACKGROUND

Epidermal growth factor receptor (EGFR) is also known as c-erbB1/HER1, whose family members are growth factor receptor tyrosine kinases, their cell surface with specific growth factors or natural ligand interactions, such as with EGF or TGF ci interactions, thereby activating the receptor tyrosine kinases. The first member of the family has been found to be a glycoprotein with apparent molecular weight of 165 KD. EGFR inhibitors include monoclonal antibodies. An anti-EGFR antibody can inhibit growth of EGFR-expressing tumor cell lines.

Glycosylation is a post-translational modification. Protein molecular surface sugar chains can have an impact on the structure and function of the protein molecules.

Glycosylation and glycan structure of a monoclonal antibody have correlation with its function, by affecting the binding of IgG molecules to FcRs, Clq and FeRn to regulate the antibody-dependent cellular cytotoxicity (ADCC), complement-dependent cytotoxicity (CDC) and half-life of IgG molecules. Glycosylation also affects the safety features of mAb, particularly non-human glycans, and has potential immunogenicity. The glycans located in Fab functional region can affect both the safety and efficacy features of these drugs.

Glycosylation is dependent on cell expression system and subclone selection, cell culture factors, such as medium components, and culture conditions. Moreover, glycosylation affects biological activity, efficacy, immunogenicity and pharmacokinetics of therapeutic proteins.

CHO cells and mouse myeloma cells (NS0, SP2/0) expression systems have been used for therapeutic antibody and Fc-fusion proteins. According to statistics, about 48% of currently approved therapeutic monoclonal antibodies are expressed in CHO cells, while 45% are expressed in murine cells (21% NS0 cells, 14% SP2/0 cells, 10% hybridoma cells).

Cetuximab (Erbitux®, C225 mab), is a recombinant chimeric monoclonal antibody specifically targeting epidermal growth factor receptor (EGFR), and was approved in many countries for the treatment of metastatic colorectal cancer and head and neck squamous cell carcinoma. However, some studies have reported that the drug hypersensitivity reactions occur at a high incidence in clinical applications. Drug specific IgE antibodies (that specifically reacts against α-Gal) were found in the serum of most patients with hypersensitivity reactions. Approved cetuximab is expressed and prepared in mammalian cells (mouse myeloma cells SP2/0). This murine cell line contains an additional α1, 3-galactosidase transferase, which primarily mediates the transfer of galactose residue is from UDP-Gal of α conformation to the terminal galactose residues, thereby generating α-Gal. α-Gal is a non-human disaccharide, found in certain glycans on mAb, especially mAb expressed in the murine cell lines. High levels of anti-α-Gal IgE antibodies were found in some patients treated with cetuximab. Further, the difference of murine cell IgG glycosylation from human is that, murine cells not only have the biosynthetic machinery to produce α-Gal epitope, but also produce N-Glycoylneuraminic acid (NGNA), rather than N-acetyl neuraminic acid (NANA). There is an additional oxygen atom in NGNA.

Glycoproteins are often associated with immunogenicity in humans if they contain NGNA residues. Some marketed therapeutic glycoproteins have cause serious adverse reactions in the patients because they contain NGNA residues. Therefore, there is a need in the art to improve the safety of cetuximab by reducing its immunogenicity. The present disclosure was made to improve drug safety.

### SUMMARY

The present disclosure provides a chimeric monoclonal antibody (CMAB009 mAb) having at least 80% NANA glycosylation terminal sialic acid at an N-glycosylation site and a glycosylation pattern of Gal-α(2,3/6)-Gal. The disclosed CMAB009 monoclonal antibody is a chimeric antibody having the same amino acid sequence (light chain/heavy chain of SEQ ID NO. 1/SEQ ID NO. 3) as cetuximab (Erbitux®) but has at least 80% NGNA terminal sialic acid and a glycosylation pattern of Gal-α(1,3)-Gal.

CMAB009 (also called STI001) has 99% of the glycosylation sialic acid is human NANA (N-Acetylneuraminic acid) with the chemical structure shown (FIG. 5). But cetuximab (Erbitux®) has 97% of the glycosylation sialic acid is human NGNA (N-Glycolyneuraminic acid) with the chemical structure shown (FIG. 5).

### DESCRIPTION OF THE FIGURES

FIG. 1 shows a comparison of the disclosed antibody CMAB009 (also called STI001) having similar binding kinetics to cetuximab (Erbitux®).

FIG. 2 shows a comparison of cellular binding to EGFR in MDA-MB476 cells of the disclosed antibody CMAB009 (also called STI001) having similar binding kinetics to cetuximab (Erbitux®).

FIG. 3 shows a comparison of cellular proliferation over doses in an IC50 of the disclosed antibody CMAB009 (also called STI001) having similar binding kinetics to cetuximab (Erbitux®). Both antibodies showed similar efficacy.

FIG. 4 shows a comparison of tumor volume over multiple doses of the disclosed antibody CMAB009 (also called STI001) having similar binding kinetics to cetuximab (Erbitux®). Both antibodies showed similar efficacy.

FIG. 5 shows that CMAB009 (also called STI001) has 99% of the glycosylation sialic acid is human NANA (N-Acetylneuraminic acid) with the chemical structure shown. But cetuximab (Erbitux®) has 97% of the glycosylation sialic acid is human NGNA (N-Glycolyneuraminic acid) with the chemical structure shown.

FIG. 6 shows peptide maps of the disclosed STI-001 and commercial cetuximab (Erbitux®) by trypsin digestion.

FIG. 7 shows Fourier Transform Infrared Spectroscopy (FT-IR), which was used to compare secondary structure of STI-001 and Erbitux over the wavelength range of 1700-1500 cm⁻¹. The spectra showed (FIG. 7) that the profiles for the three products are substantially identical, which demonstrate the structural similarities between STI-001 and Erbitux®.

FIG. 8 shows representative overlaid near UV-CD profiles, which are visually similar. Near-UV spectra of STI-001 (gray), Erbitux-US (black) and Erbitux-EU (blue).

FIG. 9 shows DSC (differential scanning calometry) scans were visually similar for STI-001 and Erbitux, indicating similar thermodynamic properties. DSC of STI-001 (black), Erbitux-US (blue) and Erbitux-EU (green).

FIG. 10 shows that the level of ADCC cytotoxic activity in the presence of the STI antibodies was as good as, if not slightly better than, Erbitux. In the absence of antibody, or with control antibody, the level of cytotoxicity was 5%.

FIG. 11 shows that the level of complement dependent cytotoxic activity in the presence of the disclosed antibodies was as good as Erbitux.

### DETAILED DESCRIPTION

The present disclosure provides safety-based therapeutic advantages when producing an anti-EGFR antibody in Chinese Hamster Ovary (CHO) cells. CMAB009 is an anti-EGFR antibody that is produced in CHO cells and has the amino acid sequence of cetuximab. In comparison to cetuximab, administration of CMAB009 to patients having cancer showed reduced immunogenicity reactions and improved efficacy, including an increase in the time in which the disease progressed.

Structurally, cetuximab has a light chain comprising the amino acid sequence set forth in SEQ ID NO: 1, and a heavy chain comprising the amino acid sequence set forth in SEQ ID NO: 3. The amino acid sequences of the cetuximab light and heavy chains are described below:

| (SEQ ID NO: 1) | |
|---|---|
| DILLTQSPVILSVSPGERVSFSCRASQSIGTNIHWYQQRTNGSPRLLIKYASESISGIPS | 60 |
| RFSGSGSGTDFTLSINSVESEDIADYYCQQNNNWPTTFGAGTKLELKRTVAAPSVFIFPP | 120 |
| SDEQLKSGTASVVCLLNNFYPREAKVQWKVDNALQSGNSQESVTEQDSKDSTYSLSSTLT | 180 |
| LSKADYEKHKVYACEVTHQGLSSPVTKSFNRGEC | |
| (SEQ ID NO: 3) | |
| QVQLKQSGPGLVQPSQSLSITCTVSGFSLTNYGVHWVRQSPGKGLEWLGVIWSGGNTDYN | 60 |
| TPFTSRLSINKDNSKSQVFFKMNSLQSNDTAIYYCARALTYYDYEFAYWGQGTLVTVSAA | 120 |
| STKGPSVFPLAPSSKSTSGGTAALGCLVKDYFPEPVTVSWNSGALTSGVHTFPAVLQSSG | 180 |
| LYSLSSVVTVPSSSLGTQTYICNVNHKPSNTKVDKRVEPKSCDKTHTCPPCPAPELLGGP | 240 |
| SVFLFPPKPKDTLMISRTPEVTCVVVDVSHEDPEVKFNWYVDGVEVHNAKTKPREEQYNS | 300 |
| TYRVVSVLTVLHQDWLNGKEYKCKVSNKALPAPIEKTISKAKGQPREPQVYTLPPSREEM | 360 |
| TKNQVSLTCLVKGFYPSDIAVEWESNGQPENNYKTTPPVLDSDGSFFLYSKLTVDKSRWQ | 420 |
| QGNVFSCSVMHEALHNHYTQKSLSLSPGK | |

As used herein, the term “CMAB009” refers to an antibody which is produced in a CHO cell. Thus, the CMAB009 antibody comprises a light chain comprising the amino acid sequence set forth in SEQ ID NO: 1 and a heavy chain comprising the amino acid sequence set forth in SEQ ID NO: 3. Further, the CMAB009 antibody does not contain either an N-glycolylneuraminic acid (NGNA) glycan or a Gal-α(1,3)-Gal glycan. The CMAB009 antibody does contain glycans associated with CHO cell expression, including, for example, a Gal-α(2, 3/6)-Gal glycan.

The glycosylation mechanism in CHO cells is similar to an IgG glycosylation mechanism in humans. The present disclosure provides a genetically engineered anti-EGFR antibody (CMAB009 mAb) with different glycan structures than cetuximab. By structure analysis, it was determined the cetuximab glycan contains a lot of α-Gal, and mostly NGNA as the terminal sialic acid. NGNA has very high immunogenicity. CMAB009 mAb glycan, by contrast, does not contain α-Gal, and terminal sialic acid is predominately in the form of NANA. The clinical trial, described herein, shows that the predominant NANA CMAB009 antibody has a good tolerance, with no drug-related hypersensitivity observed, and no IgE specific ADA was detected. At the same time of greatly reduced immunogenicity, the characteristics of CMAB009 monoclonal antibody in vivo clearance is in line with the in vivo metabolic of chimeric antibodies, and the pharmacokinetic parameters are consistent with those of cetuximab.

Compared with cetuximab monoclonal antibody, CMAB009 monoclonal antibody has the same amino acid primary structure, while does not contain α-Gal, and the terminal sialic acid is mainly N-acetylneuraminic acid (NANA). This is consistent with better tolerance that was observed in clinical studies. At the same time of greatly reduced immunogenicity, the characteristics of CMAB009 monoclonal antibody in vivo clearance is in line with the in vivo metabolic of chimeric antibodies, and the pharmacokinetic parameters are consistent with those of cetuximab.

The present disclosure provides a chimeric cetuximab-like monoclonal antibody (CMAB009 mAb) having at least 80% NANA glycosylation terminal sialic acid at an N-glycosylation site and a glycosylation pattern of Gal-α(2,3/6)-Gal. The disclosed CMAB009 monoclonal antibody is a chimeric antibody having the same amino acid sequence (light chain/heavy chain of SEQ ID NO. 1/SEQ ID NO. 3) as cetuximab (Erbitux®) which has at least 80% NGNA terminal sialic acid and a glycosylation pattern of Gal-α(1,3)-Gal.

### Comparison of CMAB009 to Cetuximab

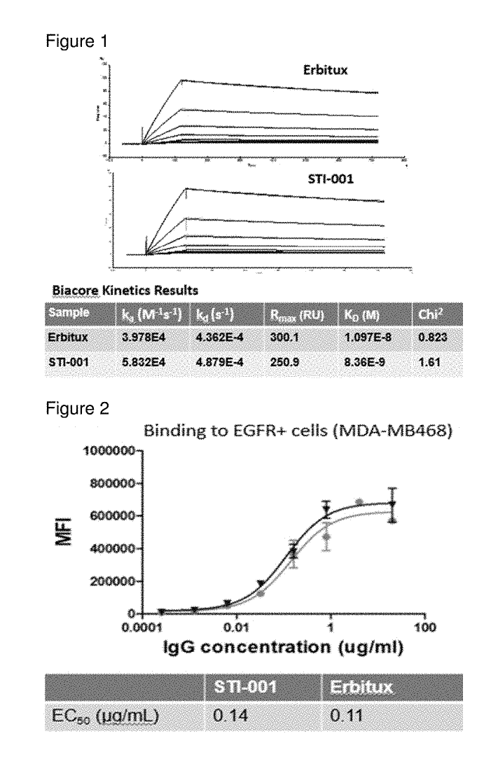

Both antibodies were compared to each other for binding, efficacy in vitro and in vivo. FIG. 1 shows a comparison of the disclosed antibody CMAB009 (also called STI001) having similar binding kinetics to cetuximab (Erbitux®) using a Biacore binding comparison. Binding to the common target EGFR was also measured in cells. FIG. 2 shows a comparison of cellular binding to EGFR in MDA-MB476 cells of the disclosed antibody CMAB009 having similar binding kinetics to cetuximab (Erbitux®).

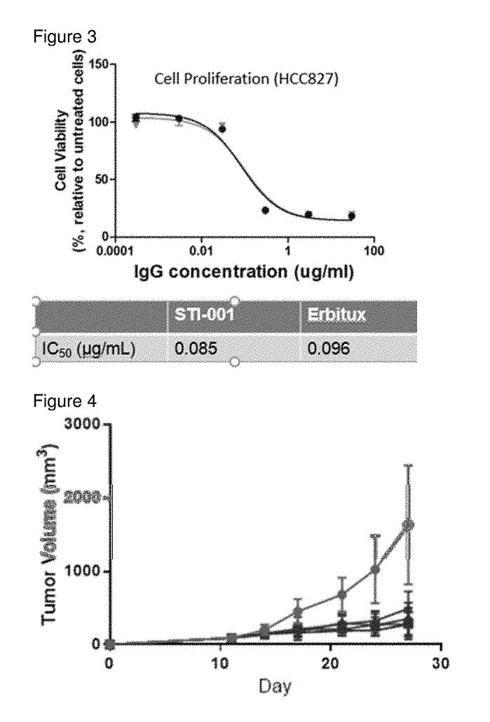

Efficacy was also measured and compared in vitro. FIG. 3 shows an in vitro comparison of cellular proliferation over doses in an IC50 of the disclosed antibody CMAB009 having similar binding kinetics to cetuximab. Both antibodies showed similar efficacy. Efficacy was also measured and compared in vivo. FIG. 4 shows an in vivo comparison of tumor volume over multiple doses of the disclosed antibody CMAB009 having similar binding kinetics to cetuximab. Both antibodies showed similar efficacy.

### Example 1: CHO Cell Production

Preferred codons of Chinese hamster were chosen. A signal peptide was selected from Chinese hamster B cell antigen receptor complex associated protein β chain.

SEQ ID NO 2
MATMVPSSVPCHWLLFLLLLFSGSS,
SEQ ID NO 4
ATGGCCACCATGGTGCCCTCTTCTGTGCCCTGCCACTGG,
and
SEQ ID NO 5
CTGCTGTTCCTGCTGCTGCTGTTCTCTGGCTCTTCT.

Liposome based co-transfection of CHO—CR-GS^{-/-} was performed and screened under pressure of CS selection system to obtain stable cell clones. After several rounds of transfection and screening, cell clones were obtained with expressing amount greater than 20 pg/cell/day.

### Example 2: Identification of Culture Conditions

A universal basal medium for CHO—CR-G5^{-/} is a chemically defined type of medium (Chemical Defined, CD). This basal medium is made by combining amino acids, vitamins, inorganic salt, glucose and trace elements according to cell growth needs and certain percentages. This basal medium met initial growth needs of the engineered cells obtained from Example 1. In order to further improve the desired antibody yield from the engineered cells, optimizations were performed for the basal medium, including adding hormones, genetically engineered recombinant growth factors, adjusting amino acids amounts.

The culture PH was: 6.5˜6.9. The expression yield of the engineered cells was greater than 30 pg/cell/day in the optimized medium, using Fed-batch culture mode.

Following purification, CMAB009 was characterized according to standard dynamic light scattering (DLS) analysis. It was determined that CMAB009 has a more homogenous size distribution in comparison to Erbitux. The z-average (z-avg) for Erbitux was determined to be 31.56 nm, while the z-avg. for CMAB009 was 16.79 nm. Furthermore, the Pdl (Polydispersity Index) of Erbitux was determined to be 0.313, versus 0.128 for CMAB009.

### Example 3: Comparison of the Glycosylation of the Culture Product

LC/MS, MS/MS techniques were used for comparative analysis of the sugar chains of CMAB009 monoclonal antibody and cetuximab (Erbitux®).

Sample preparation: Fc fragment and oligosaccharide from Fab were prepared after glucosidase digestion; oligosaccharides exonuclease treatment of oligosaccharides on Fab; 2-AB fluorescence labeling of oligosaccharides; After HILIC solid phase extraction to remove excess 2-AB, oligosaccharides were obtained with fluorescence labeled sugar chains, then analyzed via LC/MS and MS/MS chromatography.

The free glycans from glycosidase treatment of MAb, after fluorescent labeling, were analyzed by LC/MS, MS/MS and oligosaccharide exonuclease treatment. The results show that CMAB009 antibody and cetuximab each have two glycosylation sites. But Fab segments showed different glycan chain structures. The disclosed CMAB009 antibody contained at least 80% NANA glycan chain structure. However, cetuximab had at least 80% NGNA glycan chain structure. Stated otherwise, the glycans of CMAB009 Fab did not contain α-galactose, while the glycans of cetuximab Fab contained a large amount of α-galactose.

### Example 4: Clinical Tolerance Study

### Initial Evaluation of CMAB009 mAb Clinical Tolerance

An initial study enrolled a total of 18 subjects, with 3, 6, 6 subjects each assigned to dose groups of 100 mg/m² dose, 250 mg/m² dose and 400 mg/m² dose, respectively, in the study of single intravenous administration. Among the subjects enrolled in single dose study, 3 subjects withdrew due to disease progression, according to the study design the remaining 15 subjects multiple administration inclusion criteria were enrolled in the multiple dose group meeting, with 3 extra subjects were enrolled to multiple dose (Table 1)

TABLE 1
Allocation of patients to the different dose groups

| Patient No. | Single dose phase | Multiple dose phase |
|---|---|---|
| 01 | 100 mg/m² | Group A |
| 02 | 100 mg/m² | Disease progression |
| 03 | 100 mg/m² | Group A |
| 04 | 250 mg/m² | Group A |
| 05 | 250 mg/m² | Group A |
| 06 | 250 mg/m² | Group A |
| 07 | 250 mg/m² | Group A |
| 08 | 250 mg/m² | Group A |
| 09 | 250 mg/m² | Group B |
| 10 | 400 mg/m² | Group B |
| 11 | 400 mg/m² | Disease progression |
| 12 | 400 mg/m² | Group B |
| 13 | 400 mg/m² | Group B |
| 14 | 400 mg/m² | Disease progression |
| 15 | 400 mg/m² | Group B |
| 16 | — | Group B |
| 17 | — | Group B |
| 18 | — | Group B |

Subjects enrolled in this study were refractory to effective conventional treatment methods, experienced failure from conventional treatment or patients with relapse of advanced cancers, including 10 cases of colorectal cancer, 7 cases of lung cancer, 1 case of gastric cancer, the demographic statistical characteristics and prior treatment of the subjects are shown in Table 2. The multiple dose phase was designed into two groups: Group A was given 250 mg/m² of infusion once a week for four weeks. Group B was given an initial infusion of 400 mg/m², and a maintenance dose of 250 mg/m² infusion, once a week for total of 4 weeks.

TABLE 2
Patient characteristics

| | No. patients |
|---|---|
| Total | 18 |
| Treated on fixed dose extension phase | 14 |
| Median age, year range | 52 (29-64) |
| Sex | |
| male | 9 |
| female | 9 |
| Tumor type | |
| colorectal | 10 |
| NSCLC (non small cell lung cancer) | 7 |
| gastric | 1 |
| Number of prior chemotherapy regimens | |
| 2 | 7 |
| 3 | 4 |
| ≧3 | 4 |
| radiotherapy | 7 |

Comparisons and analyses were performed on subjects' baselines, and the age, height, weight, body surface, ECOG score of subjects from the three groups of single dose and the two groups of multiple doses. The results are shown in Table 3 with no statistically significant differences.

TABLE 3
Subject characteristics at baseline. Mean ± SD

| | Single dose phase | | | Multiple dose phase | |
|---|---|---|---|---|---|
| | 100 mg/m² | 250 mg/m² | 400 mg/m² | group A | group B |
| Characteristic | (n = 3) | (n = 6) | (n = 6) | (n = 7) | (n = 8) |
| Age | 58.00 | 55.00 | 55.50 | 57.00 | 54.00 |
| (years) | (31.00-61.00) | (48.00-57.00) | (42.00-61.00) | (48.00-61.00) | (49.00-58.00) |
| Height (cm) | 161.00 | 168.50 | 173.00 | 165.00 | 171.00 |
| | (145.00-170.00) | (165.00-172.00) | (160.00-176.00) | (152.00-172.00) | (160.00-175.00) |
| Weight (kg) | 54.00 | 67.50 | 63.50 | 67.00 | 66.50 |
| | (37.00-58.00) | (67.00-70.00) | (54.00-81.00) | (46.00-71.00) | (61.75-76.00) |
| BSA | 1.55 | 1.75 | 1.75 | 1.74 | 1.76 |
| (m²) | (1.20-1.67) | (1.70-1.80) | (1.50-1.90) | (1.40-1.81) | (1.61-1.87) |
| ECOG | 1.00 | 1.00 | 1.00 | 1.00 | 1.00 |
| | (1.00-2.00) | (1.00-1.00) | (1.00-1.00) | (1.00-2.00) | (1.00-1.00) |

The results showed that the CMAB009 monoclonal antibody was well tolerated. Among the 18 subjects, there was no grade III-IV drug-related toxicity as showed in Table 4, with all occurring drug-related toxicity at grade I-II, and the incidence of toxicity was independent of the doses or the dosing frequency. No dose-limiting toxicity was observed, and no drug-related hypersensitivity was observed.

TABLE 4
CMAB009-related toxicities

| | CTC Grade | | | | |
|---|---|---|---|---|---|
| N = 18 | I | II | III-IV | Total no events | % |
| Acne-like rash | 10 | 1 | 0 | 11 | 61.1 |
| Fever chills | 6 | 4 | 0 | 10 | 55.6 |
| Nausea/vomiting | 5 | 0 | 0 | 5 | 27.8 |
| Headache | 3 | 0 | 0 | 3 | 16.7 |
| Fatigue/melaise | 1 | 0 | 0 | 1 | 5.6 |
| transaminase elevation | 1 | 0 | 0 | 1 | 5.6 |
| paronychia | 1 | 0 | 0 | 1 | 5.6 |
| nasal discharge | 1 | 0 | 0 | 1 | 5.6 |

There was no CMAB009 antibody related hypersensitivity observed in this study. The published the incidence of hypersensitivity reactions associated with cetuximab, by contrast, reached 31%, including class III-IV hypersensitivity incidence of 13%. Among 76 subjects who received cetuximab treatment, 25 subjects had hypersensitivity, with hypersensitivity incidence reached 33% (Chung et al. “Cetuximab-induced anaphylaxis and IgE specific for galactose-alpha-1,3-galactose” New Engl. J. Med. 2008; 358 (11): 1109-17).

### Example 5: Clinical Result Safety, Immunogenicity Study

CMAB009 monoclonal antibody clinical safety were most adverse events were drug-related rash. There was no clinically significant new toxicity observed, and there no was severe hypersensitivity observed among 73 subjects studied.

In this study, a biosensor was made with biofilm interference technology and optical fibers, in which the bottom was covered with SA ligands conjugated with biomolecule compatible layers. Once the captured biotinylated antibody was bound to its ligand, biofilm thickness increased and reflected light interference spectral curve drifted a measurable distance. Thus, a real-time measurement of intermolecular interactions was made. This method is equivalent to a self-assembly process of the captured antibodies, which formed a range of optimal conformations at a certain density for capturing antibody on the surface of the biosensor, which not only improves the analytical sensitivity but also increases the linear range, which helps reduce the false-positive reactions from non-specific binding.

As to the immunogenicity analysis of CMAB009 monoclonal antibody in this study, the results showed that there was ADA (Anti-drug antibody) detected in 1.4% ( 1/73) of the subjects, with IgG type confirmed by subtype analysis. This was not an IgE type ADA mediated by hypersensitivity. The results of this study were consistent with the results of clinical safety evaluation, since there were no severe hypersensitivity reactions observed among subjects in clinical studies.

### Example 6: CMAB009 Treatment Results in Improved Efficacy for Treating Cancer and Reduced Immunogenicity

CMAB009 was administered to patients having metastatic colorectal cancer in a Phase 2/3 study to determine the efficacy and immunogenicity of CMAB009. As described below, the results from the study were then compared to similar studies performed using cetuximab. Surprisingly, it was determined that CMAB009 has additional efficacy beyond that known for cetuximab. For example, CMAB009 was able to increase the overall survival and length of time to disease progression in patients. The below study is comparable to the cetuximab study described in Cunningham et al. (2004) New Eng. J. Med. 351:337-345.

The CMAB900 study was initiated by screening patients to identify those with EGFR positive, metastatic colorectal cancer. 501 patients were identified and randomized in a 2:1 manner to group 1 or group 2. Group 1 included 337 patients who were administered a combination of CMAB009 and irinotecan. Specifically, the patients in group 1 were administered an initial dose of 400 mg/m² of CMAB009 followed by weekly infusions of 250 mg/m² thereafter. Irinotecan doses were maintained according to each patient's pre-trial therapy. Group 2 included 164 patients who were administered irinotecan monotherapy at a dose consistent with the patient's therapy prior to the study. Patients in both groups were treated until the disease progressed or the patient reached an unacceptable level of toxicity. Patient baseline characteristics are provided in Table 5.

TABLE 5
Baseline characteristics

| | CMAB009 Ph2/3 trial | |
|---|---|---|
| | CMAB009 + | Irinotecan |
| | irinotecan | monotherapy |
| | (n = 337) | (n = 164) |
| Age (yr) | | |
| Median | 55 | 55 |
| Range | 20-72 | 20-71 |
| Sex-no. (%) | | |
| Male | 195 (57.9) | 104 (63.4) |
| Female | 142 (42.1) | 60 (36.6) |
| Race-no. (%) | | |
| White | 0 | 0 |
| Black | 0 | 0 |
| Asian | 334 (99.1) | 159 (97.0) |
| Others | 3 (0.9) | 5 (3.0) |

Patients were evaluated for radiologic response in both group 1 and group 2. The results are described in Table 6. Note the overall response rate (ORR) in Table 6 was determined according to the sum of the rate of CR and PR, and the disease control rate (DCR) was determined according to the sum of the rates of CR, PR, and SD.

When compared to data reported for cetuximab from a similar study (see Cunningham et al. (2004) New Eng. J. Med. 351:337-345), patients receiving CMAB900 showed better overall survival (8.6 months for patients receiving cetuximab+irinotecan vs. 17.6 months for patients receiving CMAB900+irinotecan) and an increased time to disease progression (4.1 months for patients receiving cetuximab+irinotecan vs, 6.6 months for patients receiving CMAB900+irinotecan).

TABLE 6
Radiologic response of CMAB009

| | CMAB009 Ph2/3 trial | | |
|---|---|---|---|
| | CMAB009 + | Irinotecan | |
| | irinotecan | monotherapy | |
| | (n = 337) | (n = 164) | P-value |
| Complete response | 4 (1.2) | 1 (0.6) | |
| Partial response | 107 (31.8) | 20 (12.2) | |
| Stable disease | 159 (47.2) | 86 (52.4) | |
| Progressive disease | 47 (13.9) | 44 (26.8) | |
| Unable to evaluate | 20 (5.9) | 13 (7.9) | |
| Overall response rate | 111 (32.9 [27.9- | 21 (12.8 [8.1- | <0.001 |
| | 38.2]) | 18.9]) | |
| Disease control rate | 270 (80.1 [75.5- | 107 (65.2 [57.4- | 0.0004 |
| | 84.2]) | 72.5]) | |
| Overall survival | 17.5 | 16.8 | |
| (months) | | | |
| Time to disease | 6.6 | 4.1 | |
| progression (months) | | | |

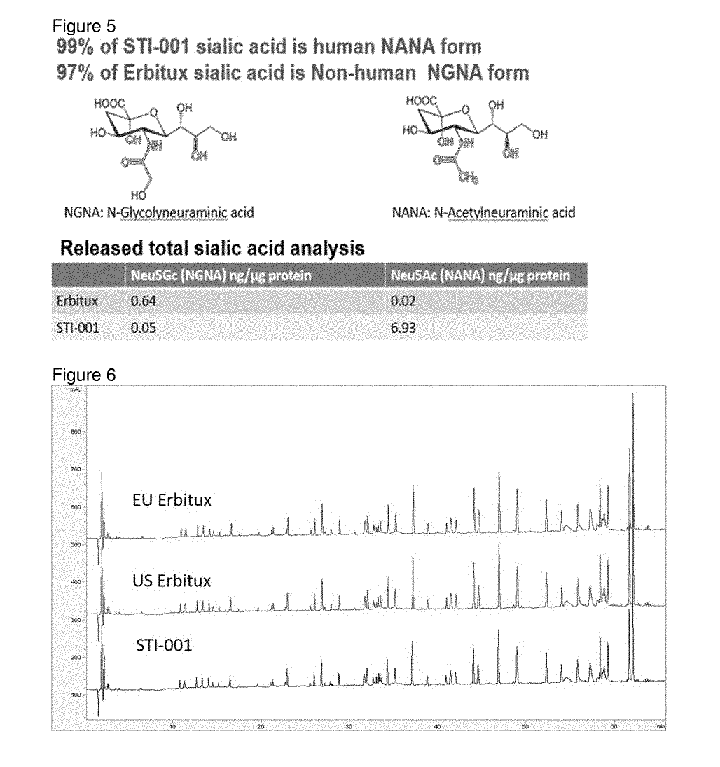

When compared to reported data for cetuximab, surprisingly the overall survival of the patients was greater in the patients receiving CMAB009, i.e., 8.6 months for cetuximab+irinotecan vs. 17.5 months for CMAB009+irinotecan. Data showing an increase in disease progression from this CMAB009 study is also provided in FIG. 5 (compare to published cetuximab data; see FIG. 2 of Cunningham et al. (2004) New Eng. J. Med. 351:337-345). Data showing an increase in overall survival from this CMAB009 study is also provided in FIG. 6 (compare to published cetuximab data; see FIG. 3 of Cunningham et al. (2004) New Eng. J. Med. 351:337-345).

A safety evaluation of the study is provided below in Table 7.

TABLE 7
Safety evaluation

| | Antibody + | | | |
|---|---|---|---|---|
| | Irino | Irino | Total | |
| | (N = 342) | (N = 170) | (N = 512) | PValue |
| At least one AE | 332 (97.1%) | 148 (87.1%) | 480 (93.8%) | <.0001 |
| At least one ADR | 320 (93.6%) | 126 (74.1%) | 446 (87.1%) | <.0001 |
| At least one | 302 (88.3%) | 120 (70.6%) | 422 (82.4%) | <.0001 |
| important AE | | | | |
| At least one Level □ | 38 (11.1%) | 12 (7.1%) | 50 (9.8%) | 0.1458 |
| or above AE | | | | |
| At least one Level □ | 27 (7.9%) | 10 (5.9%) | 37 (7.2%) | 0.4076 |
| or above ADR | | | | |
| At least one Level □ | 37 (10.8%) | 11 (6.5%) | 48 (9.4%) | 0.1119 |
| or above important | | | | |
| AE | | | | |
| At least one SAE | 23 (6.7%) | 8 (4.7%) | 31 (6.1%) | 0.3669 |
| At least one test | 7 (2.0%) | 1 (0.6%) | 8 (1.6%) | 0.2800 |
| drug-related SAE | | | | |
| At least one AE | 73 (21.3%) | 27 (15.9%) | 100 (19.5%) | 0.1420 |
| which lead to stop | | | | |
| drug treatment | | | | |
| Test drug-related | 1 (0.3%) | 0 | 1 (0.2%) | 1.0 |
| death AE | | | | |

Notably, adverse events from the CMBA009 study were lower than those reported for cetuximab. The grade 3-4 adverse events are described below in Table 8 (compare to Table 4 of Cunningham et al. (2004) New Eng. J. Med. 351:337-345).

TABLE 8
Grade 3-4 adverse events for CMBA009 study.

| | STI001 + | | Irinotecan | |
|---|---|---|---|---|
| | irinotecan | | monotherapy | |
| | (n = 337) | | (n = 164) | |
| Any | 180 | (54.2) | 57 | (38.9) |
| Anemia | 3 | (0.9) | 3 | (1.8) |
| Neutropenia | 53 | (15.7) | 14 | (8.5) |
| Thrombocytopenia | 0 | | 0 | |
| Diarrhea | 35 | (10.4) | 12 | (7.3) |
| Asthenia | 19 | (5.6) | 6 | (3.7) |
| Acne-like rash | 0 | | 0 | |
| Nausea and vomiting | 16 | (4.7) | 16 | (9.8) |
| Abdominal pain | 3 | (0.9) | 0 | |
| Stomatitis | 1 | (0.3) | 0 | |
| pnea | N/A | | N/A | |
| Fever | 5 | (1.5) | 1 | (0.9) |

In sum, despite having the same primary structure, CMAB900 surprisingly was not only more effective than cetuximab, providing, for example, a longer time to disease progression, but had a reduced rate of adverse events associated with hypersensitivity reactions, e.g., acne-like rash or diarrhea.

### Example 7: CMAB009 Treatment Results i

This example describes a comparison of physicochemical, biochemical, and functional properties of two anti-EGFR Cetuximabs, STI-001 and Erbitux®. Results from these studies disclose that STI-001 and Erbitux® possess the same amino acid sequence, primary structure, secondary and tertiary structure and thermal stability. Comparative biological activities, product related size variants, aggregate and particle levels were similar. STI-001 has different charge variants and glycosylation patterns than Erbitux, which is caused by using a different host cell expressing system. STI-001 has over 99% human sialic acid form NANA. Erbitux has majority non-human type sialic acid NGNA.

Experiments presented in this example aim to assess analytical similarity between STI-001 and two cetuximab reference products using the state-of-the-art analytical methods. The comparison studies used seven batches of STI-001, three batches of Erbitux-US and three batches of Erbitux-EU. Biochemical and biophysical in vitro characterization of STI-001 and US and EU licensed Erbitux®. STI-001 was manufactured by Mabtech Inc. and Erbitux was purchased on US market and EU market. The materials and equipment used in these studies are summarized in Table.

TABLE 8
Materials and Equipment Used

| Equipment/ | | | |
|---|---|---|---|
| Instrument Name | Equipment ID | Serial Number | Source |
| Agilent 1260 HPLC | EQN1590 | NA | Agilent |
| Agilent 1290 UHPLC | EQN1592 | | Agilent |
| Agilent bioanalyzer | EQN1594 | DE13806785 | Agilent |
| Q-TOF LC-MS | Waters LC-MS | YCA234 | Waters |
| Protein Simple iCE3 (icIEF) | EQN1593 | JW0740/10339 | Protein Simple |
| MicroCal VP-DSC | | SYS12901 | Malvern |
| Dynamic light scattering | DLS | MAL1087922 | Malvern |
| IntelliCyt ® HTFC screening | IntelliCyt | 3286 | NA |
| system | | | |
| Biacore T200 | Biacore | 1950211 | NA |
| Flexstation 3 Multimode | NA | 05933 | NA |
| Microplate Reader | | | |
| Hypercyt | Model C6 | NA | NA |
| Microscope | CK2 | NA | NA |
| Countess ® II Automated | AMQAX1000 | 1615 185A 213 | NA |
| Cell Counter | | | |
| Culture cell Incubator | Model 3326 | 35166-6207 | NA |
| Centrifuge | 5702 | | NA |
| Centrifuge Sorvall RT6000 | NA | 820 6005 | NA |
| Refrigerated | | | |

| Raw Material Name | |
|---|---|
| (Reagents/Standards/Chemicals) | Mfg. Catalog # |
| IAM | Sigma/I1149 |
| DTT | Thermo Scientific/20290 |
| Trpsin Endoproteinase | Thermo Scientific/90055 |
| Water | Fisher/W6-4 |
| DPBS | Corning Cell Grow 21-031-CM |
| NAP-5 column | GE/17-0853-02 |
| Waters CSH 130 C18 | Waters/186006938 |
| Formic acid | Fisher/A117-50 |
| Guanidine-HCl | Pierce/24115 |
| Tris-HCl | SIGMA/T6666 |
| 0.5M EDTA pH 7.5 | Fisher/BP2482 |
| Acetonitrile | Fisher/LS120-4 |
| 4-20% Tris-glycine gel | Thermo Fisher/EC6025BOX |
| Agilent protein 230 Kit | Agilent/5067-1517 |
| Potassium phosphate, monbasic | J. T. Baker/3052-01 |
| Potassium phosphate, dibasic | J. T. Baker/4012-01 |
| Potassium chloride | J. T. Baker/4008-01 |
| Pharmalyte pH 3-10 | GE/17045601 |
| pI marker 6.61 | ProteinSimple/102409 |
| pI marker 9.50 | ProteinSimple/101996 |
| Rapid PNGase F | NEB/P0710S |
| Cell Dissociation Buffer | Gibco/13151-014 |
| PBS | Corning-CellGro/21-031-CM |
| MDA-MB-468 | NCI/507784 |
| Assay Plate, 96 well, no lid, V-bottom, non-treated | Costar/3897 |
| Anti-human IgG-PE | Southern Biotech/2040-09 |
| RPMI 1640 Medium, GlutaMAX ™ Supplement | Lif Technologies/61870-036 |
| Premium-grade Fetal Bovine Serum, 100% US origin | Seradigm/1500-500 |
| Costar ™ 96-Well White Clear-Bottom Plates | Fisher/07-200-587 |
| CellTiter-Glo ® Luminescent Cell Viability Assay | Promega/G7572 |

Reducing and non-reducing capillary electrophoresis sodium dodecyl sulphate (CE-SDS) were performed on the cetuximab samples using Agilent Bioanalyzer 2100 following the manufacturer's instructions. Size exclusion chromatography (SEC) was performed on cetuximab samples using a TSKgel SuperSW mAb HR column (4 μm, 7.8 mm×300 mm) and a mobile phase of 0.2 M potassium phosphate, 0.25 M potassium chloride, pH 6.2, at a flow rate of 0.8 mL/min. A 50 μg sample was loaded onto the column. Data were monitored and collected at 280 nm by an ultraviolet (UV) detector.

The charge variants of STI-001 and Erbitux samples were determined by icIEF (Imaging Capillary Isoelectric Focusing) using an iCE3 Analyzer with a fluorocarbon (FC) coated capillary cartridge. The ampholyte solution consisted of 4% Pharmalyte pH 3-10, 0.35% (V/V) methyl cellulose, 1M urea with 0.7% (V/V) of each of the pI markers 6.61 and 9.50. The cetuximab samples were diluted to 2 mg/mL with DI water and then mixed with ampholyte solution at a 3:17 (V/V) ratio. The analyte was 80 mM phosphoric acid, and the catholyte was 100 mM sodium hydroxide, both in 0.1% MC. Once loaded, the sample was focused by introducing a potential of 1,500 volts for one minute, followed by potential of 3,000 volts for 6 minutes. An image of focused cetuximab was obtained by passing 280 nm UV light through the capillary and into the lens of a charge coupled device (CCD) digital camera.

DSC (Differential scanning calorimetry) experiment was done on a MicroCal VP-DSC. Samples were diluted to 1 mg/mL in formulation buffer and was degassed for 10 minutes before analysis. The reference cell was filled with formulation buffer. The sample was heated from 20° C. to 90° C. at a heating rate of 60° C./hour. The pre-scan was 15 minutes, the filtering period was 10 seconds, and the feedback mode/gain was set to passive. The midpoint of a thermal transition temperature (Tm, or thermal transition temperature) was obtained by analyzing the data using Origin 7 software.

For reduced protein analysis of Erbitux and STI-001 (deglycosylated), antibody (20 μg) was first treated with FabRICATOR to cleave the protein at the hinge region. The resulting solution was then treated with Rapid PNGase F buffer at 80° C. for 10 min for denaturation and reduction, and with Rapid PNGase F at 50° C. for 15 min for deglycosylation.

For LCMS analysis, an aliquot (5 μg) sample was injected onto a Waters Acquity column (BEH300 C4, 1.7 μm, 2.1×150 mm, 80° C. column temperature). Antibody was eluted from the column with a 12 min gradient (25-40% B, 0.4 mL/min flow rate). For reduced antibody analysis, all experiments were performed on a Waters H class UPLC system coupled to a Waters Xevo G2 TOF mass spectrometer. The mass spectrometer was operated in positive ion, sensitivity mode with detection in the range of 500-4000 m/z. Source parameters were as follows: capillary voltage, 3.0 kV; sampling cone voltage, 40.0 V; source temperature, 125° C.; desolvation temperature, 350° C.; cone gas flow, 10 L/hr; desolvation gas flow, 800 L/hr. The protein peak was deconvolved by MassLynx MaxEnt1 function according to the following parameters: output resolution, 2.0 Da/channel; uniform Gaussian width at half height, 0.8 Da for intact antibody, 0.5 Da for reduced antibody; minimum intensity ratios 33% for left and right; maximum number of iteration of 20.

For reducing peptide mapping with trypsin, the antibody was diluted with denaturation buffer (6M GuHCl, 360 mM Tris, ImM EDTA, pH 8.6) to 1 mg/ml, reduced with DTT (final concentration 5 mM, 80° C. for 15 min) and alkylated with iodoacetamide (final concentration 15 mM, 37° C. for 15 min). The sample was then exchanged into digestion buffer (25 mM Tris, ImM CaCl₂, pH 8.3). The sample in digestion buffer was digested with Trypsin for 4 hours at 37° C. The digestion was then quenched with formic acid to get a final concentration of 0.2% (v/v).

The peptides were then analyzed on a Waters UPLC coupled online with Q-TOF Mass Spectrometer. An aliquot (10 μg) sample was injected onto an Agilent AdvanceBio peptide mapping column (C18, 2.7 μm, 2.1×150 mm). Antibody was eluted from the column with a gradient of 0-19% in 30 min, 19%-27% in 18 min and 27-51% in 27 min, the flow rate was 200 μL/min, and the column temperature was set to 45° C. Mobile phase A was 0.1% formic acid and mobile phase B was 0.1% formic acid in acetonitrile.

FTIR experiment was performed by HTL Biosolution Inc., Camarillo, Calif. In brief, FTIR spectra were collected on a JASCO 4200 FTIR spectrometer equipped with a room temperature TGS detector and a sATR device. Spectra were collected at a 4 cm⁻¹ resolution with data average of 256 scans. Residual moisture peaks were also subtracted from the spectra of samples before further analysis. FTIR spectra of buffers have also been collected and the spectrum of buffer has been subtracted from that of the protein sample. After subtraction of buffer spectrum, residual moisture peaks were also subtracted from all spectra collected. Finally, second derivative spectra were calculated by using the Savitzky-Golay method, with a 2^{nd} order of polynomial function and the number of convolution point is 13.

CD experiments were performed by Alliance Protein Laboratories, San Diego, Calif. Antibody samples were diluted with formulation buffer to 1 mg/ml for near UV CD. CD measurements were carried out at room temperature on a Jasco J-715 spectropolarimeter using 1 cm cell. After subtracting buffer spectrum, the CD spectrum of the protein was converted to the mean residue ellipticity (CD intensity per amino acid) using the protein concentration, the mean residue weight (average weight per amino acid) of 109.85 and the path-length of the cell.

Dynamic light scattering (DLS) measurements were made on a Malvern ZEN3600 at room temperature. The scattered light was detected at an angle of 90°. For N-linked oligosaccharide analysis, N-glycans were released from 200 μg of protein under denatured condition using PNGase F followed by purification of the glycans using SPE cartridges (C18 and PGC). N-linked glycans were labeled with 2-Ab and detected by UPLC-FLR and mass spectrometer. For total sialic acid analysis, sialic acid analysis was performed by UCSD (University of California, San Diego) Glycotechnology Core Facility, La Jolla, Calif. The antibody sample was dissolved in a final concentration of 2 M acetic acid and heated to 80° C. for 3 hours to release sialic acids. The released sialic acids were collected by filtrating through an ultra-10 filter with 3,000 MWCO, dried and analysed by HPAEC-PAD using a Dionex CarboPac PA-1 column eluted with a sodium acetate gradient that separates N-acetylneuraminic acid and N-glycolylneuraminic acid.

A cell binding assay provided MDA-MB-468 triple-negative breast cancer (TNBC) cells highly expressing EGFR harvested with enzyme-free Cell Dissociation Buffer (GIBCO) and transferred to V-Bottom 96 well-plates (50,000 cells/well). Cells were incubated on ice for 45 min with serial dilutions of STI-001 antibody in FACS buffer (PBS+2% FBS). After 2 washes in FACS buffer, a 1:1000 dilution of Phycoerythrin-conjugated anti-Human IgG was added and incubated for 20 min. Following a final wash, fluorescence intensity was measured on the Hypercyt High Throughput Flow Cytometer (HTFC, Intellicyt). Data were analysed using Graphpad Prism software and non-linear regression fit. Data points are shown as the median fluorescence intensity (MFI) of positively labeled cells +/−Standard Error. EC₅₀ values are reported as the concentration of antibody to achieve 50% of maximal binding to the cells.

A cell proliferation assay provided EGFR-expressing cells (MDA-MB-468) in log phase were lifted and resuspended in RPMI+1% FBS to 55,555 cells/ml. Cells were seeded in white 96-Well Clear Bottom plates (5,000 cells/well in 90 ul). On the same day, serial dilutions of the antibodies were prepared in RPMI+1% FBS, in 10× premixes, and added to cells (10 μl/well) in triplicate. After 3 days incubation at 37° C., cell proliferation was analysed as follows: 100 ul of Cell Titer Glo buffer (Promega) was added to each well. Plates were incubated with shaking at room temperature for 20 min. Luminescence signal was then measured on a Flexstation 3 plate reader. Data were reported as Relative Luminescence Units. Dose-response curves were generated in GraphPad prism, and IC₅₀ values were calculated using non-linear regression fit (Log (inhibitor) vs. response—Variable slope equation).

Anti-human Fc antibody (GE, BR-1008-39) was immobilized on CM5 sensor chip (Biocore) to approximately 1000RU using standard NHS/EDC coupling methodology. Antibodies (about 5 μg/mL) were captured for 60 s at a flow rate 10 μL/min. Recombinant human EGFR/His was serially diluted in running buffer (HBS-EP). All measurements were conducted in HBS-EP buffer with a flow rate of 30 μL/min. The antibody was diluted appropriately to obtain a series of concentrations. A 1:1 (Langmuir) binding model was used to fit the data. The experiment was run on a GE Biacore T200.

A triple negative breast cancer cell line, pre-incubated with or without test antibodies, was cultured with natural killer cells (effector cells). After an appropriate period, the amount of tumor cell lysis was determined.

Preparation of effector cells (NK cells): Peripheral blood mononuclear cells were prepared from blood obtained from a San Diego blood bank using SepMate-50 tubes (Cat.#15450) and Lymphoprep (Cat.#0780), both from Stemcell technologies. From this, NK cells were isolated using EasySep negative selection “Human NK cell Enrichment Kit” Cat.#19055 also from Stemcell Technologies. The resultant NK cell (>95% pure) population were then cultured in RPMI medium supplemented with 10% fetal calf serum plus interleukin 2 (Prospec, Ness Ziona, Israel) at 100 U/ml. The next day, cells were harvested, washed and resuspended in fresh RPMI+10% FCS.

Preparation of target cells: EGFR positive, triple negative, breast cancer cell line MDA-MB-468 were maintained in RPMI+10% FCS. On the day of assay, the cells were removed from the culture flask with the aid of cell dissociation buffer (Gibco cat#13151-014). Once cells had detached, they were washed and resuspended in fresh RPMI+10% FCS.

Assay performance: Target cells were added to the wells of a flat bottom white plate (Costar cat#3917) at 5×10³ per well. Test antibodies at 10 microgram/ml or medium was added to appropriate wells. After 20 minutes at 37° C., the plate is washed followed by the addition of the effector cells (1.5×10⁵ per well) to give an effector to target ratio of 30:1. After 4 hours at 37° C., substrate from the CytoTox-Glo kit (Promega Cat.# G9291) was added and the plate processed according to the instructions provided in the kit.

An anti-EGFR binding ELISA assay used a direct binding ELISA format. Recombinant human EGFR-His (Cat#10001-H08H, lot# LC08DE1601, Sino Bio) was adsorbed onto a microtiter plate followed by blocking of the plate and addition of anti-EGFR mAb dilutions (2.5-fold serial dilution with starting concentration 10 ng/mL of mAb). An HRP (horse radish peroxidase)-labelled anti-Human IgG reagent binds the anti-EGFR mAb. Finally, a substrate (TMB) was added which is converted by HRP to a visible colour. The HRP enzyme reaction was then stopped with acid and the colour signal is detected by a plate reader. The amount of anti-EGFR bound was directly proportional to the amount of colour generated. Wash steps were included between incubations to remove excess unbound reagents after each step prior to substrate addition. EC50 values of the mAbs were calculated in Prism 7 by plotting A450 versus the mAb concentrations and fitting the curves with the sigmoidal dose-response analysis (four parameters fit).

Antibody STI-001 was compared, head-to-head, with Reference Medicinal Product (RMP)—Erbitux® sourced from EU and US market. The lot information is provided in Table 8.

TABLE 8
Reference Medicinal Product Lot information

| Product/Strength | Lot Number | Manuf. Date | Exp. Date | Source |
|---|---|---|---|---|
| STI-001 (20 mg/mL) | aDS-20160623 | June 2016 | June 2019 | Sorrento |
| STI-001 (20 mg/mL) | aDS-20160702 | July 2016 | July 2019 | |
| STI-001 (18 mg/mL) | aDS-20160711 | July 2016 | July 2019 | |
| STI-001 (10 mg/mL) | 20160201 | February 2016 | February 2019 | |
| STI-001 (10 mg/mL) | Clinical trial Lot | June 2010 | May 2013 | |
| | T20100601 | | | |
| STI-001 (10 mg/mL) | Clinical trial Lot | June 2010 | May 2013 | |
| | T20100602 | | | |
| STI-001 (10 mg/mL) | Clinical trial Lot | July 2010 | June 2013 | |
| | T20100701 | | | |
| Erbitux ® (2 mg/mL) | IMD246 | NA | January 2017 | US |
| Erbitux ® (2 mg/mL) | IMF411 | NA | July 2018 | |
| Erbitux ® (2 mg/mL) | IMF417 | NA | October 2018 | |
| Erbitux ® (5 mg/mL) | 214376 | NA | October 2019 | EU |
| Erbitux ® (5 mg/mL) | 212461 | NA | September 2019 | |
| Erbitux ® (5 mg/mL) | 210683 | NA | September 2019 | |

The comparative characterization results are summarized in tables 9 and 10. The primary and higher-order structure of STI-001, as well as size variants are similar to Erbitux (US) and Erbitux (EU) RMPs. The aggregate, particle levels, and product purity between STI-001 and Erbitux RMPs are similar. Furthermore, functional biological characterization, including proliferation bioassay, antigen binding assay, effector functions such as ADCC and CDC, FcγR binding assays revealed that STI-001 has equivalent biological activities to Erbitux (US) and Erbitux (EU). STI-001 has shown different glycosylation patterns with Erbitux RMPs. STI-001 has predominant human sialic acid form N-Acetylneuraminic acid (NANA). Erbitux has predominant non-human sialic acid form N-Glycolyneuraminic acid (NGNA).

TABLE 9
Summary of Comparability Assessment Results

| Method | | Result |
|---|---|---|
| Identity; | Peptide mapping | STI-001 has the same amino acid sequence for both |
| Primary | and Amino acid | heavy chain and light chain as those for Erbitux ® |
| structure | sequencing | based on amino acid sequencing analysis of peptides |
| | | fragments after digestions with trypsin protease. |
| | Reduce and | Same molecular weights for separated heavy chain |
| | deglycosylated HC | and light chain based on the deglycosylated heavy |
| | and LC by LC-MS | chain and light chain; |
| Higher order | Secondary structure | STI-001 exhibits the same spectrum by FTIR, |
| structure | by FT-IR | demonstrating the same secondary structure as that |
| | | of Erbitux ®. |
| | Tertiary structure | STI-001 exhibits the same spectrum by Near-UV |
| | by Near-UV CD | CD, demonstrating the same tertiary structure as that |
| | | of Erbitux ®. |
| | Thermal stability | STI-001 has the same thermal stability profile as that |
| | by DSC | of Erbitux ®. |
| Potency | Antigen binding | STI-001 has the same binding affinity to EGFR as |
| | affinity by Biacore | that of Erbitux ®. |
| | Antigen binding | STI-001 has the same binding affinity to EGFR as |
| | assay ELISA | that of Erbitux ®. |
| | Cell based Antigen | Based on the binding assay of STI-001 and of |
| | binding assay | Erbitux ® to MDA-MB468 an EGFR expressing |
| | | breast cancer cell line, the EC₅₀ for both products |
| | | are comparable. |
| | Cell proliferation | Based on the ability of STI-001 and of Erbitux ® to |
| | assay | inhibit the cell proliferation of HCC827 cell line, a |
| | | lung cancer cell line, the IC₅₀ for both products are |
| | | comparable. |
| | FcγR binding | STI-001 and Erbitux (US/EU) was bound to |
| | affinity | recombinant FcγRI with similar affinity. |

TABLE 10
Summary of Quality Comparability Results of STI-001
Compared with Erbitux ® Authorized US and EU.

| Method | | Result |
|---|---|---|
| Potency | ADCC | The level of cytotoxic activity in the |
| | | presence of the STI-001 antibodies |
| | | was as good as Erbitux US and EU. |
| | CDC | The level of cytotoxic activity in the |
| | | presence of the STI-001 antibodies |
| | | was as good as Erbitux US and EU. |
| Purity (Particle | SEC-HPLC | The purity of STI-001 and Erbitux ® |
| size; | | as determined by SEC-HPLC, are |
| aggregates) | | comparable. The content of monomer |
| | | is >98% for both products. |
| | Dynamic Light | STI-001 exhibits similar size |
| | Scattering | distribution and polydispersity index |
| | (DLS) | (PdI) as those of Erbitux. |
| | CE-SDS | The purity of STI-001 and Erbitux ® |
| | | as determined by CE-SDS, are |
| | | comparable. |
| Charge | icIEF | STI-001 has a different charge profile |
| variants | | with a slight shift to more basic |
| | | species for Erbitux ®, which is |
| | | consistent with a lower level of |
| | | sialic acid observed in Erbitux ®. |
| Glycan profile | N-linked release | Erbitux has more complex glycan |
| | oligosaccharide | profiles than STI-001. Erbitux ® |
| | | has non-human glycans, which may |
| | | induce immunogenicity/ |
| | | hypersensitivity. |
| | Sialic acid | STI-001 has only human sialic acid, |
| | analysis | NANA (N-Acetylneuraminic acid) |
| | | while Erbitux ® has only Non- |
| | | human sialic acid NGNA |
| | | (N-Glycolyneuraminic acid). |

Antibody amino acid sequence identifications were performed by trypsin peptide mapping coupled with LC-MS analysis on the intact molecular of STI-001 and Erbitux. There were identical amino acid sequences. FIG. 6 shows representative chromatographic profiles. The data demonstrated that STI-001 has a matching chromatographic profile to that of US commercial Erbitux and EU commercial Erbitux. No additional peptides or missing peptides were detected in the comparison between the three products.

The similarity for the primary structures between STI-001 and Erbitux (US) and Erbitux (EU) was also investigated using reduced and deglycosylated HC and LC mass analysis. Results of HC and LC analysis are shown in Table 11, which provided further assurance that polypeptide compositions were similar measuring molecular weight.

TABLE 11
Summary of Reduced and deglycosylated
HC and LC mass analysis

| | | Light | Heavy | Heavy |
|---|---|---|---|---|
| | | Chain | Chain | Chain |
| | | MW | Fc MW | Fab MW |
| Product | Lot Number | (Da) | (Da) | (Da) |
| STI-001 | aDS-20160623 | 23425 | 25452 | 23789 |
| | aDS-20160702 | 23425 | 25452 | 23789 |
| | aDS-20160711 | 23426 | 25451 | 23789 |
| | 20160201 | 23425 | 25452 | 23789 |
| | T20100601 | 23425 | 25451 | 23788 |
| | T20100602 | 23425 | 25452 | 23788 |
| | T20100701 | 23425 | 25453 | 23789 |
| Erbitux ® (US) | IMD246 | 23425 | 25451 | 23788 |
| | IMF411 | 23425 | 25450 | 23788 |
| | IMF417 | 23425 | 25451 | 23788 |
| Erbitux ® (EU) | 214376 | 23425 | 25450 | 23788 |
| | 212461 | 23424 | 25450 | 23788 |
| | 210683 | 23425 | 25450 | 23788 |

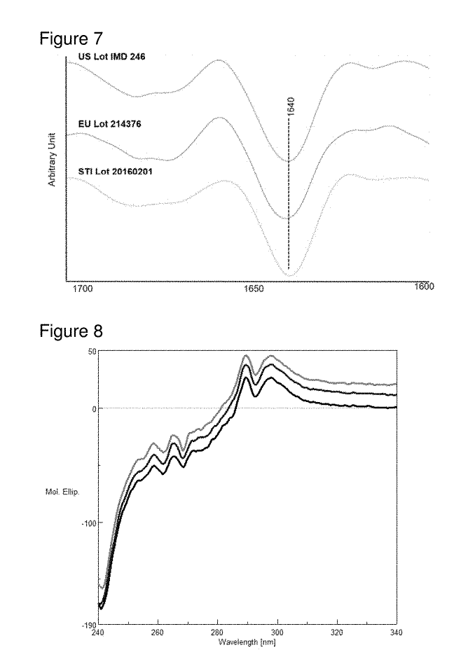

Fourier Transform Infrared Spectroscopy (FT-IR) was used to compare secondary structure of STI-001 and Erbitux over the wavelength range of 1700-1500 cm⁻¹. The spectra showed (FIG. 7) that the profiles for the three products are substantially identical, which demonstrate the structural similarities between STI-001 and Erbitux®.

The tertiary structure of the antibodies was determined by near-UV circular dichroism (CD). CD spectra of a protein in the “near-UV” spectral region (250-350 nm) can be sensitive to certain aspects of tertiary structure. At these wavelengths the chromophores are the aromatic amino acids and disulfide bonds, and the CD signals they produce are sensitive to the overall tertiary structure of the protein. The near UV-CD spectral of all individual STI-001 lots were similar compared with Erbitux. FIG. 8 shows representative overlaid near UV-CD profiles, which are visually similar.

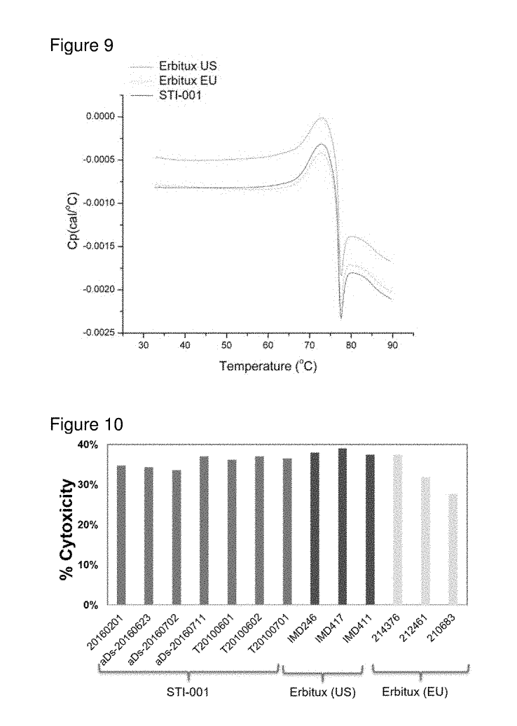

The thermodynamic properties of STI-001 and Erbitux (US and EU licensed) products were assessed and compared by DSC (differential scanning calometry). The DSC scans were visually similar for STI-001 and Erbitux RMPs (FIG. 9), and the thermal melting temperatures were also similar among the three products (Table 12), indicating similar thermodynamic properties.

TABLE 12
Summary of Tm (melting temperature) values

| Product | Lot Number | Tₘ (° C.) |
|---|---|---|
| STI-001 | aDS-20160623 | 72.5 |
| | aDS-20160702 | 72.8 |
| | aDS-20160711 | 73.1 |
| | 20160201 | 72.8 |
| | T20100601 | 72.8 |
| | T20100602 | 72.6 |
| | T20100701 | 72.9 |
| Erbitux ® (US) | IMD246 | 72.9 |
| | IMF411 | 73.0 |
| | IMF417 | 72.7 |
| Erbitux ® (EU) | 214376 | 72.7 |
| | 212461 | 72.9 |
| | 210683 | 72.5 |

Antigen binding was measured to determine binding affinity of anti-EGFR antibodies (STI-001 and Erbitux US and Erbitux EU) to recombinant EGFR/His. This was measured by Biacore. The Kd values are reported in Table 13. The anti-EGFR antibody Erbitux (US and EU) and ST-001 showed similar kinetic properties for their binding to human EGFR protein as shown in Table 13.

TABLE 13
Comparison of binding affinity between
STI-001 and Erbitux US and EU

| | | ka | kd | Rmax | KD |
|---|---|---|---|---|---|
| Source | Lot | (1/Ms) | (1/s) | (RU) | (M) |
| Erbitux | IMD246 | 9.87E4 | 1.48E−4 | 84.22 | 1.50E−9 |
| (US) | IMD417 | 7.63E4 | 1.75E−4 | 80.59 | 2.29E−9 |
| | IMD411 | 5.52E4 | 1.41E−4 | 56.91 | 2.54E−9 |
| Erbitux | 214376 | 8.57E4 | 1.38E−4 | 138.60 | 1.61E−9 |
| (EU) | 212461 | 7.79E4 | 1.63E−4 | 103.50 | 2.10E−9 |
| | 210683 | 7.78E4 | 1.65E−4 | 96.89 | 2.12E−9 |
| STI-001 | aDS-20160623 | 8.67E4 | 1.01E−4 | 82.14 | 1.17E−9 |
| | aDS-20160702 | 5.49E4 | 1.95E−4 | 275.5 | 3.55E−9 |
| | aDS-20160711 | 5.07E4 | 1.97E−4 | 280.6 | 3.88E−9 |
| | 20160201 | 5.40E4 | 1.86E−4 | 247.8 | 3.44E−9 |
| | T20100601 | 1.16E5 | 1.54E−4 | 62.92 | 1.32E−9 |
| | T20100602 | 6.72E4 | 1.65E−4 | 92.46 | 2.45E−9 |
| | T20100701 | 1.94E5 | 2.38E−4 | 73.12 | 1.23E−9 |

STI-001, Erbitux (US/EU) were titrated in a standard ELISA against cognate antigen and detected using an HRP-labeled secondary antibody to measure antigen binding affinity by ELISA. Table 14 shows that the EC50 values are similar between the antibody lots.

TABLE 14
Relative binding affinity by ELISA

| | | Relative binding |
|---|---|---|
| Source | Lot | affinity |
| Erbitux (US) | IMD246 | 120% |
| | IMD417 | 123% |
| | IMD411 | 100% |
| Erbitux (EU) | 214376 | 100% |
| | 212461 | 103% |
| | 210683 | 111% |
| STI-001 | aDS-20160623 | 110% |
| | aDS-20160702 | 109% |
| | aDS-20160711 | 100% |
| | 20160201 | 101% |
| | T20100601 | 99% |
| | T20100602 | 112% |
| | T20100701 | 103% |

A cell-based antigen binding assay measured binding of STI-001 and Erbitux with MDA-MB-468 cells, a triple negative breast cancer cell line highly expressing EGFR.

According to results presented in Table 15, no significant differences were observed between STI-001 and Erbitux (US and EU). The antibodies bound to cellular antigen with the comparable affinity.

TABLE 15
EC50

| Source | Lot | EC50 (nM) |
|---|---|---|
| Erbitux (US) | IMD246 | 0.70 |
| | IMD417 | 0.47 |
| | IMD411 | 0.70 |
| Erbitux (EU) | 214376 | 0.56 |
| | 212461 | 0.41 |
| | 210683 | 0.47 |
| STI-001 | aDS-20160623 | 1.32 |
| | aDS-20160702 | 1.52 |
| | aDS-20160711 | 1.53 |
| | 20160201 | 0.25 |
| | T20100601 | 0.46 |
| | T20100602 | 0.35 |
| | T20100701 | 0.37 |

A cell proliferation assay measured anti-proliferative effects of STI-001 and Erbitux (US and EU) in a cell proliferation assay. Briefly MDA-MB-468 cells were grown for 3 days in the presence of increasing amounts of either STI-001 or Erbitux, and cell viability was then measured. The results, shown in Table 16, indicate that STI-001 and Erbitux display very similar inhibitory activity on cell proliferation.

TABLE 16
IC50

| Source | Lot | Relative IC50 |
|---|---|---|
| Erbitux (US) | IMD246 | 92% |
| | IMD417 | 87% |
| | IMD411 | 100% |
| Erbitux (EU) | 214376 | 92% |
| | 212461 | 94% |
| | 210683 | 105% |
| STI-001 | aDS-20160623 | 93% |
| | aDS-20160702 | 46% |
| | aDS-20160711 | 155% |
| | 20160201 | 66% |
| | T20100601 | 100% |
| | T20100602 | 128% |
| | T20100701 | 87% |

STI-001 or Erbitux (US/EU) was bound to recombinant FcγRI in a standard ELISA in increasing concentrations. The EC50 values were derived from 4PL curve analysis. The results show that the EC50 values of the antibodies are all similar between STI-001 and Erbitux.

TABLE 17
FcγR Relative binding affinity

| | | Relative binding |
|---|---|---|
| Source | Lot | affinity |
| Erbitux (US) | IMD246 | 82% |
| | IMD417 | 86% |
| | IMD411 | 100% |
| Erbitux (EU) | 214376 | 130% |
| | 212461 | 124% |
| | 210683 | 82% |
| STI-001 | aDS-20160623 | 122% |
| | aDS-20160702 | 119% |
| | aDS-20160711 | 111% |
| | 20160201 | 89% |
| | T20100601 | 91% |
| | T20100602 | 97% |
| | T20100701 | 91% |

STI-001 antibodies were compared with Erbitux EU and Erbitux US for their ability to support ADCC. For these studies, the triple negative EGFR expressing breast cancer cell line, MDA-MB-468 was cultured with natural killer (NK) cells in the presence or absence of test antibody at 10 μg/ml. Measurement of cytotoxicity was performed after four hours of incubation using a luciferase based kit from Promega. FIG. 10 shows that the level of ADCC cytotoxic activity in the presence of the STI antibodies was as good as, if not slightly better than, Erbitux. In the absence of antibody, or with control antibody, the level of cytotoxicity was 5%.

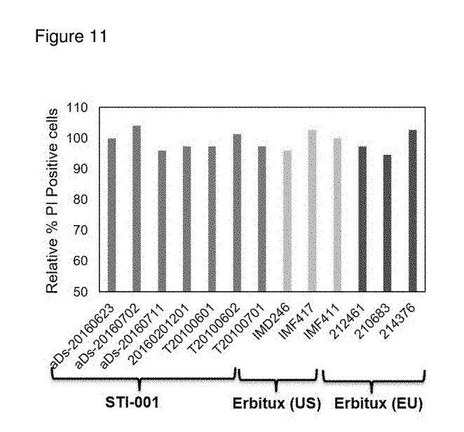

STI-001 antibodies were compared with Erbitux EU and Erbitux US for their ability to support complement dependent cytotoxicity (CDC). For these studies, the triple negative EGFR expressing breast cancer cell line, MDA-MB-468 was incubated with or without the test antibodies added at 10 μg/ml. After 20 minutes, baby rabbit complement was added as a 10% v/v solution. To evaluate cytotoxicity, propidium iodide was added after a one hour incubation and the percentage dead cells quantitated by flow cytometry. FIG. 11 shows that the level of complement dependent cytotoxic activity in the presence of the disclosed antibodies was as good as Erbitux.

Product monomer content was determined by SEC-HPLC (Table 18). Both STI-001 and Erbitux (US) and Erbitux (EU) exhibited predominant monomer content (>97%). These results confirmed that STI-001 and Erbitux (US) and Erbitux (EU) are similar regards to monomer purity.

TABLE 18
Summary Result of SEC-HPLC

| | | % | % | % |
|---|---|---|---|---|
| Source | Lot | HMWS | Main Peak | LMWS |
| Erbitux (US) | IMD246 | 0.3 | 98.1 | 1.6 |
| | IMF411 | 1.5 | 97.2 | 1.3 |
| | IMF 417 | 1.5 | 97.5 | 1.0 |
| Erbitux (EU) | 210683 | 0.7 | 97.9 | 1.4 |
| | 212461 | 0.6 | 98.0 | 1.4 |
| | 214376 | 1.3 | 97.4 | 1.3 |
| STI-001 | 20160201 | 1.0 | 97.7 | 1.3 |
| | aDS 20160711 | 0.6 | 97.6 | 1.8 |
| | aDS 20160702 | 0.7 | 97.4 | 1.9 |
| | aDS 20160623 | 1.4 | 96.3 | 2.3 |
| | T20100601 | 1.7 | 95.2 | 3.1 |
| | T20100602 | 1.8 | 96.3 | 1.9 |
| | T20100701 | 1.9 | 95.7 | 2.4 |

Dynamic light scattering (DLS) is a technique that can measure the size of particles down to 1 nm in diameter. Particles in suspension undergo random Brownian motion. If these particles are illuminated with a laser beam, the intensity of the scattered light fluctuates at a rate that is dependent upon the size of the particles. Analysis of these intensity fluctuations yields the velocity of the Brownian motion and hence the particle size. Table 19 shows intensity size distributions obtained from 3 repeat measurements of STI-001 and Erbitux samples. STI-001 and Erbitux have similar main size distribution.

TABLE 19
Comparison of particle size between STI-001,
Erbitux (US) and Erbitux (EU)

| | | Z-Avg. | |
|---|---|---|---|
| Source | Lot | (d · nm) | PdI |
| Erbitux (US) | IMD246 | 20.0 | 0.19 |
| | IMF411 | 11.9 | 0.07 |
| | IMF 417 | 11.6 | 0.04 |
| Erbitux (EU) | 210683 | 15.2 | 0.10 |
| | 212461 | 15.8 | 0.13 |
| | 214376 | 15.4 | 0.11 |
| STI-001 | 20160201 | 14.1 | 0.06 |
| | aDS 20160711 | 15.3 | 0.14 |
| | aDS 20160702 | 15.9 | 0.11 |
| | aDS 20160623 | 15.6 | 0.07 |
| | T20100601 | 13.5 | 0.06 |
| | T20100602 | 13.5 | 0.08 |
| | T20100701 | 13.5 | 0.07 |

CE-SDS was used to assess the product purity. STI-001 and Erbitux (US and EU) were analyzed under non-reducing and reducing conditions. Protein markers with known molecular weights and sample were run in parallel. CE-SDS showed STI-001 and Erbitux (US and EU) have the same purity under both reducing and non-reducing conditions (Table 20).

TABLE 20
Summary of CE-SDS Result

| | | Non- | | | |
|---|---|---|---|---|---|
| | | reducing | Reducing Condition | | |
| | | Main Band | HC | LC | Sum |
| Source | Lot | % | % | % | (HC + LC) |
| Erbitux | IMD246 | 97.4 | 64.2 | 35.7 | 99.9 |
| (US) | IMF411 | 98.3 | 64.1 | 35.6 | 99.9 |
| | IMF 417 | 98.1 | 64.4 | 35.7 | 100.0 |
| Erbitux | 210683 | 98.2 | 66.5 | 33.4 | 99.9 |
| (EU) | 212461 | 98.3 | 64.8 | 34.8 | 99.6 |
| | 214376 | 98.6 | 65.6 | 34.4 | 100.0 |
| STI-001 | 20160201 | 97.4 | 68.6 | 31.5 | 100.0 |
| | aDS 20160711 | 96.9 | 66.0 | 31.7 | 97.7 |
| | aDS 20160702 | 97.6 | 67.7 | 32.1 | 99.8 |
| | aDS 20160623 | 97.5 | 68.2 | 30.0 | 98.2 |
| | T20100601 | 93.4 | 66.4 | 33.6 | 100.0 |
| | T20100602 | 94.4 | 66.5 | 33.2 | 99.7 |
| | T20100701 | 94.8 | 66.1 | 33.4 | 99.4 |

Charge variants in STI-001 and Erbitux were analyzed by imaging capillary isoelectric focusing (icIEF). STI-001 and Erbitux RMPs were expressed by using different host cells. STI-001 used CHO cell system and Erbitux used SP2/0 cell lines. The processes are also different. Therefore, the charge variants are expected to be different. However, their main pIs and charge variants percentages were very similar.

### 1.1 Glycan Analysis

In addition to the conserved Fc glycosylation at Asn 297, cetuximab also contains another N-linked glycosylation site at V_{H} domain Asn 99. To compared the glycan heterogeneity associated with STI-001 and Erbitux RMPs, several methods were used including LC-MS, released glycans mass spectrometry and total sialic acid analysis.

Fc and Fab glycosylations were released by PNGase F and labelled with 2-AB (2-aminobenzamide). 2-Ab labelled glycans are analyzed by UPLC and TOF-MS. The UPLC chromatograms showed STI-001 and Erbitux have different glycan profiles. Erbitux showed more oligosaccharide species than STI-001. Table 21 lists all the glycan forms identified and their relative abundances.

The types of amounts of sialic acids capping the glycan structures display by STI-001 and Erbitux were assessed. Sialic acid was detected in the major form of N-glycolylneuraminic acid (NGNA) for Erbitux and N-acetyl-neuraminic acid (NANA) for STI-001 (Table 22). The predominant sialic acid made by human is NANA. NGNA has been shown to have potential contribution to immunogenicity/hypersensitivity in human. STI-001 has human sialic acid form NANA. Erbitux has non-human type sialic acid NGNA.

TABLE 21
Total sialic acid comparison between STI-001 and Erbitux RMPs

| | Source | Lot | | NGNA % | | | | NANA% | |
|---|---|---|---|---|---|---|---|---|---|
| | Erbitux (US) | IMD246 | | 100% | | | | 0% | |
| | | IMF411 | | 100% | | | | 0% | |
| | | IMF 417 | | 100% | | | | 0% | |
| | Erbitux (EU) | 210683 | | 100% | | | | 0% | |
| | | 212461 | | 100% | | | | 0% | |
| | | 214376 | | 100% | | | | 0% | |
| | STI-001 | 20160201 | | 0% | | | | 100% | |
| | | aDS 20160711 | | 0% | | | | 100% | |
| | | Glycan species | | | | | | | |
| | | | | | | | G2F- | | |
| | | | | | | | Gal1/G2 | G2- | G2F- |
| Source | Lot NO | G0F | G0FB | G1F | G1F' | G2F | F-Gal2 | NGNA | NGNA |
| US | IMD246 | 27.1% | 0.4% | 17.8% | 5.3% | 4.5% | 16.6% | 0.4% | 7.8% |
| | IMF411 | 28.8% | 0.2% | 18.9% | 5.8% | 4.7% | 13.8% | 0 | 7.6% |
| | IMF417 | 25.8% | 0.5% | 18.6% | 5.5% | 4.9% | 16.5% | 0 | 6.7% |
| EU | 214376 | 23.8% | 0.2% | 16.8% | 5.0% | 4.3% | 18.8% | 0.8% | 8.8% |
| | 212461 | 25.6% | 0 | 18.3% | 5.5% | 4.7% | 17.6% | 0.4% | 8.5% |
| | 210683 | 26.0% | 0 | 18.2% | 5.4% | 4.7% | 17.1% | 0 | 8.8% |

| | | |
|---|---|---|
| aDS 20160702 | 0% | 100% |
| aDS 20160623 | 0% | 100% |
| T20100601 | 0% | 100% |
| T20100602 | 0% | 100% |
| T20100701 | 0% | 100% |

In conclusion, the foregoing analytical comparisons show that the STI-001 and Erbitux® (US and EU) antibodies possess the same amino acid sequence, primary structure, secondary and tertiary structure and thermal stability, very comparable purity, impurity, biochemical, and functional properties. But STI-001 has different charge variants and glycosyulation patterns with Erbitux (US and EU). STI-001 has over 99% human sialic acid form NANA. Erbitux has majority non-human type sialic acid NGNA, which is believed to induce immunogenicity/hypersensitivity in humans.

1. An anti-EGFR antibody pharmaceutical composition comprising a light chain amino acid sequence set forth in SEQ ID NO: 1 and a heavy chain amino acid sequence set forth in SEQ ID NO: 3,
wherein the anti-EGFR antibody has a z-average (z-avg) of about 10-25 nm as determined by dynamic light scattering (DLS) analysis, and
wherein the anti-EGFR antibody comprises a Gal-α(2, 3/6)-Gal glycan.

2. The anti-EGFR antibody pharmaceutical composition of claim 1, wherein the z-avg of the antibody is 15-20 nm.

3. The anti-EGFR antibody pharmaceutical composition of claim 1, wherein the the sialic acid glycosylation is at least 80% NANA glycosylation terminal sialic acid at an N-glycosylation site.

4. A chimeric anti-EGFR monoclonal antibody having at least 80% NANA glycosylation terminal sialic acid at an N-glycosylation site and a glycosylation pattern of Gal-α(2,3/6)-Gal.
